(19) United States
(12) Patent Application Publication
(10) Pub. No.: US 2009/0004232 A1
(43) Pub. Date: Jan. 1, 2009

(54) BIOACTIVE PHARMACEUTICAL/COSMETIC COMPOSITIONS AND MIXED SOLUBILIZATION PROCESS FOR THE FORMULATION THEREOF

(75) Inventor: ALAIN BRZOKEWICZ, Antibes (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 12/119,038

(22) Filed: May 12, 2008

Related U.S. Application Data
(63) Continuation of application No. PCT/FR2006/051156, filed on Nov. 9, 2006.

(30) Foreign Application Priority Data
Nov. 10, 2005 (FR) 0511448

(51) Int. Cl. A61K 9/107; A61K 8/06; A61K 9/12; A61K 31/56; A61K 31/59; A61P 17/06

(52) U.S. Cl. 424/401; 424/400; 424/45; 514/181; 514/167

(57) ABSTRACT

Physically stable pharmaceutical/cosmetic compositions contain at least one hydrophilic phase, at least one lipophilic phase and at least one active principle, e.g., at least one steroidal anti-inflammatory agent, anti-fungal agent, anti-parasitic agent, nuclear receptor modulator and/or sex steroid, said at least one active principle being present in dissolved state in each of these phases; these are formulated via characteristic multi-step methodology.

### CROSS-REFERENCE TO PRIORITY/PCT APPLICATIONS

This application claims priority under 35 U.S.C. § 119 of FR0511448, filed Nov. 10, 2005, and is a continuation of PCT/FR 2006/051156, filed Nov. 9, 2006, and designating the United States (published in the French language on May 24, 2007 as WO 2007/057598 A2; the title and abstract were also published in English), each hereby expressly incorporated by reference in its entirety and each assigned to the assignee hereof.

### BACKGROUND OF THE INVENTION

1. Technical Field of the Invention

The present invention relates to compositions for pharmaceutical or cosmetic application, comprising at least two phases dissolved in which is one and the same active principle, and also to the method for preparing such compositions.

2. Description of Background and/or Related and/or Prior Art

It is common in pharmacy or in the cosmetics field to produce and use emulsions in which the active principle is dissolved in a single phase, whether this be a hydrophilic phase or a lipophilic phase.

However, the solubilization of an active principle in a single phase is not free of problems. Effectively, the maximum concentration of the active principle, which can be dissolved in one phase, may be below the concentration that is therapeutically effective in the targeted pathology or pathologies.

Migration phenomena of the active principle from its solvating phase towards the other phase may, in addition, appear and lead to its recrystallization. In this case, the recrystallization may prove particularly problematic when the active principle has an irritant potential. Problems of irritation may be encountered, due to the direct contact of the crystals with the skin, during application of the formulation.

The same recrystallization problems may be manifested when the active principle is dispersed in a phase that is very weakly solvating for this active principle.

The expression “active principle in dispersed form” means an active principle in the form of solid particles, suspended in a given carrier. Preferably, the dispersed particles are visible to the naked eye or by using optical microscopy such as crossed-polarization microscopy. Thus, the size of the dispersed particles is preferably greater than or equal to 1 micron.

In order to improve skin penetration, U.S. Pat. No. 6,491,396 describes an oil-in-water (O/W) emulsion within which the active principle, maxacalcitol, vitamin D3 derivative, is present in the lipophilic phase and/or in the hydrophilic phase. Maxacalcitol is then in a dissolved state in the lipophilic phase, and in a dispersed state in the hydrophilic phase.

Such an emulsion is not completely satisfactory. In particular, the drawbacks linked to the dispersion in a carrier are multiple. Thus, beyond a certain concentration, problems of aggregates and of heterogeneity of the dispersion may appear. The search for a dispersant suitable for the active principle is additionally not always apparent. The particle size of the active principle must also be the subject of much attention depending on the application site and the intended biological target.

On the other hand, the presence, in the formulation, of the active principle in two different states (dissolved in one phase and dispersed in the other) may prove prejudicial. This is because the dispersed active principle forms a recrystallization seed and may result in a problem of crystal growth, a phenomenon that is difficult to control and damaging to the formulation in terms of physical stability and therapeutic efficacy.

### SUMMARY OF THE INVENTION

It has now been determined that it is possible to solve the problems mentioned above by solubilization of the active principle in both phases of the composition. In particular, it has now been shown that, surprisingly, although the active principle is dissolved in both phases, the composition is physically stable and it is thus possible to attain a higher overall concentration of the therapeutically effective active principle.

The expression “physical stability” of the compositions according to the invention means, in particular, compositions which do not exhibit a phase shift over time after storage for three months at 4° C., at ambient temperature and at 40° C. According to the invention, another physical stability parameter observed is, in particular, the absence of recrystallization phenomenon at ambient temperature and at 4° C., over three months.

Specifically, at the therapeutically effective concentration, no recrystallization phenomenon of the active principle was observed after storing the formulations for three months at ambient temperature and at 4° C.

In the present text, the expression “active principle in dissolved form” means a molecular state dispersion in a liquid, no crystallization of the active principle being visible to the naked eye nor even using a crossed polarization optical microscope, or using other more accurate techniques such as electron microscopy. Preferably, the bioactive principle particles have a size below 1 nm.

Preferably, according to the invention, the expression “active principle in dissolved form” means a molecular state dispersion in a liquid for which no crystallization of the active principle is visible to the naked eye or using an optical microscope.

The present invention therefore features pharmaceutical or cosmetic compositions comprising at least two phases having different polarities, one and the same active principle present in dissolved form in each of these phases, and also to the method for formulating such compositions.

This invention thus features pharmaceutical or cosmetic compositions comprising at least one hydrophilic phase, at least one lipophilic phase and at least one active principle, said active principle being present in dissolved form in at least said hydrophilic phase and at least said lipophilic phase.

### DETAILED DESCRIPTION OF BEST MODE AND SPECIFIC/PREFERRED EMBODIMENTS OF THE INVENTION

Preferably, the subject compositions are emulsions.

Preferably, the subject compositions comprise one hydrophilic phase (as one only) and one lipophilic phase (and one only).

According to the invention, the solubilization of the active principle in the two phases of the composition presents multiple advantages.

It makes it possible to adjust the release of the active principle. For example, in an O/W (oil-in-water) emulsion, the fraction of the active principle which is dissolved in the outer phase of the emulsion (here, the hydrophilic phase) is released before the fraction dissolved in the inner phase (here, the lipophilic phase). This deferred release of the active principle thus provides the formulation a role as reservoir of the active principle.

The solubilization in the various phases makes it possible, furthermore, to reduce the problems of irritation, when the active principle is potentially an irritant. On the one hand, the recrystallization phenomenon of the active principle is considerably reduced and, on the other hand, the adjustment of its release makes it possible to substantially improve the tolerance of a formulation for topical application by avoiding a rapid accumulation of the active principle in the skin layers, responsible for the irritation problems. The solubilization of the active principle in both phases of the composition makes it possible to attain a concentration of active principle greater than that obtained if the latter was dissolved solely in a single phase. This concentration of active principle dissolved in a single phase very often proves to be below the therapeutically effective concentration. The solubilization of the active principle in the different phases of the composition makes it possible to attain a sufficient concentration to elicit a known efficacy in the targeted pathology or pathologies.

Active Principles:

Any bioactive principle may be employed, it being understood that it must be soluble and be in the same molecular state in both of the phases of the emulsion. This is because it is known that certain active principles may be present in the dissolved state in the various phases of an emulsion, but in salified form in one and in non-salified form in the other. However, the active principle dissolved in both phases of the composition according to the invention is present in the same molecular state.

Furthermore, in the compositions according to the invention, the active principle is present in the same physical state in both phases of the composition. Specifically, according to the invention, the active principle is dissolved in each of the phases of the composition and under no circumstances is dispersed in one or other of the two phases.

The molecules employed as the active principle in these compositions therefore preferably have a particular solubility profile described below.

They have a sufficient solubility in water or the polar compounds conventionally used in the pharmaceutical or cosmetic field, preferably in topical formulations.

This solubility in the polar media enables them to be dissolved in the hydrophilic phase of the composition.

On the other hand, these molecules have a sufficient solubility in the lipophilic excipients customarily used in the pharmaceutical or cosmetic field, preferably in formulations for topical application, that allow them to be dissolved in the lipophilic phase of the emulsion.

The expression “sufficient solubility” means a solubility from 0.005% to 1%, preferably from 0.01% to 0.5% by weight of active principle relative to the total weight of the solvent (water, polar compounds, lipophilic excipients).

The term “soluble” means the characteristic of certain solid substances to form solutions when they are introduced into a suitable solvent. Various parameters may be involved in the solubility of a molecule in the solvent in question. Thus, the molecular weight, the melting point, the chromatographic purity, the crystal structure, but also the chemical structure, and especially the nature of the functional groups and of the hydrophilic or lipophilic parts, contribute to its solubility profile.

The parameters that enable the solubility profile of a molecule to be characterized are, in particular, the solubility parameter and the log P of the molecule.

The solubility parameter is all of the solvent/solute molecular interactions, namely the dispersion forces, the polar interactions and hydrogen bonding type interaction.

The Hansen method, known to one skilled in the art, makes it possible to determine the overall solubility parameter of a molecule theoretically (use of the group contribution method applied to the molecule) and experimentally, by evaluation of the solubility of this molecule in solvents whose overall solubility parameter has been determined.

The advantage of this method is to establish a list of potential solvents for the molecule studied.

These potential solvents have a solubility parameter close to that of the molecule.

The overall solubility parameter of the molecules present in the compositions of the invention ranges from 10 to 40, preferably from 15 to 30 and more preferably from 18 to 24.

Another parameter that makes it possible to evaluate the solubility of a molecule is its log P value. The log P value of a molecule is represented by the ratio of its solubility in octanol to its solubility in water.

This parameter is important for molecules administered topically.

This is because this octanol/water partition is representative of the partition of the molecule during its application to the skin, a partition from the hydrophilic parts (cells) on the one hand and lipophilic parts (intercellular cement) of the skin on the other hand.

The logarithm of the partition coefficient (denoted by log P) then makes it possible to know its polarity. Specifically, the higher the log P value, the more the molecule will have a lipophilic character. Conversely, the lower this value is, the more the molecule will have a polar character. The molecules that can be used in the present invention have a log P value from 2 to 6, and preferably from 4 to 5, so as to be able to be dissolved in the hydrophilic phase and in the lipophilic phase of the composition.

Moreover, the molecules employed as the active principle are preferably chemically stable in each of the phases in which they are dissolved.

Steroidal anti-inflammatory agents, anti-fungal agents, anti-parasitic agents, nuclear receptor modulators and sex steroid hormones are non-exhaustive examples of molecules that have such physicochemical properties.

Among the steroidal anti-inflammatory agents, exemplary are the corticosteroids such as, for example, beclomethasone, betamethasone, clobetasol, cortisone, desonide, dexamethasone, fluochloronide, fluocinolone acetonide, fluocinonide, fludroxycortide, formocortal, halcinonide, hydrocortisone, methylprednisolone, prednisone, triamcinolone, triamcinolone acetonide or mixtures thereof.

Among the anti-fungal agents, exemplary are amorolfine, clotrimazole, fluconazole, ketoconazole, miconazole or else tolnaftate.

Among the anti-parasitic agents, exemplary is ivermectin and more generally the family of avermectins.

The sex steroid hormones according to the invention include DHEA or dehydroepiandrosterone, and also its chemical and/or biological precursors and derivatives.

Among the nuclear receptor modulators, exemplary are the retinoids and their analogues, the modulator compounds of PPAR (Peroxisome-Proliferator Activated Receptor) receptors and vitamin D and its analogues or derivatives.

Among the retinoids, exemplary are retinoic acid or tretinoin, 13-cis-retinoic acid or isotretinoin, retinol, acitretin, adapalene, or mixtures thereof.

Among the PPAR receptor modulator compounds, exemplary are thiazolidinediones and derivatives thereof.

The expression “vitamin D analogues” means the various forms of vitamin D such as, for example, vitamin D₂ or vitamin D₃. The expression “vitamin D derivatives” means the compounds that have biological properties similar to those of vitamin D, especially transactivation properties for vitamin D response elements (VDREs), such as an agonist or antagonist activity towards receptors of vitamin D or its derivatives. These compounds are not generally natural metabolites of vitamin D. They are, in particular, synthetic compounds comprising the vitamin D backbone with modifications on the side chains and/or that also comprise modifications in the backbone itself. Compounds derived from vitamin D that can be used according to the invention thus comprise structural, for example diaromatic, analogues.

By way of illustration of these vitamin D derivatives, exemplary are, in particular, calcipotriol, calcitriol or 1,25-dihydroxyvitamin D₃, doxercalciferol, secalcitol, maxacalcitol, seocalcitol, tacalcitol, paricalcitol, falecalcitriol, 1α,24(S)-dihydroxyvitamin D₂, 1(S),3(R)-dihydroxy-20(R)-[((3-(2-hydroxy-2-propyl)phenyl)methoxy)methyl]-9,10-secopregna-5(Z),7(E),10(19)-triene, mixtures thereof and derivatives thereof.

According to one particular embodiment, the vitamin D derivatives preferably employed according to the invention are described in WO 00/26167. These are compounds that are structural analogues of vitamin D which show a selective activity on cell proliferation and differentiation without having a hypercalcemic character.

These compounds may be represented by the general formula (I) below:

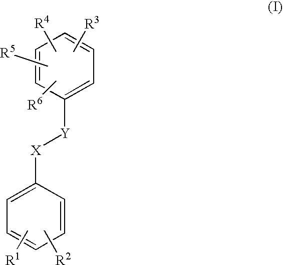

in which:

R¹ is a hydrogen atom, a methyl radical or a —(CH₂)ₙ—OR⁷ radical;

R² is a —(CH₂)ₙ—OR⁸ radical,

n, R⁷ and R⁸ are as defined below;

X—Y is a bond selected from the bonds of formulae (a) to (d) below which may be read from left to right or vice versa:

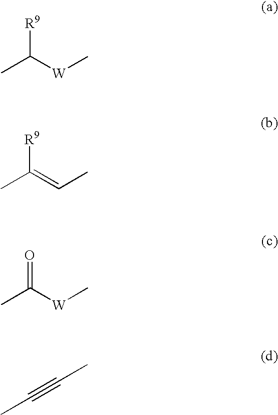

R⁹ and W are as defined below,

R³ is the chain of vitamin D₂ or of vitamin D₃,

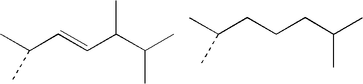

the dotted lines represent the bond connecting the chain to the benzene ring represented in figure (I); or

R³ is a chain having from 4 to 8 carbon atoms substituted by one or more hydroxyl groups, the hydroxyl groups optionally being protected in acetoxy, methoxy or ethoxy, trimethylsilyloxy, tert-butyldimethylsilyloxy or tetrahydropyranyloxy form and optionally in addition:

substituted by one or more lower alkyl or cycloalkyl groups; and/or

substituted by one or more halogen atoms; and/or

substituted by one or more CF₃ groups; and/or

in which one or more carbon atoms of the chain are replaced by one or more oxygen, sulfur or nitrogen atoms, with the proviso that the nitrogen atoms may optionally be substituted by lower alkyl radicals; and/or

in which one or more single bonds of the chain are replaced by one or more double and/or triple bonds;

R³ being positioned on the benzene ring para or meta to the X—Y bond;

R⁴, R⁵ and R⁶, which may be identical or different, are each a hydrogen atom, a lower alkyl radical, a halogen atom, a —OR¹⁰ radical or a polyether radical,

R¹⁰ is as defined below;

n is 0, 1 or 2;

R⁷ and R⁸, which may be identical or different, are each a hydrogen atom, an acetyl radical, a trimethylsilyl radical, a tert-butyldimethylsilyl radical or a tetrahydropyranyl radical;

R⁹ is a hydrogen atom or a lower alkyl radical;

W is an oxygen or sulfur atom, a —CH₂— radical or a —NH— radical which may optionally be substituted by a lower alkyl radical; and

R¹⁰ is a hydrogen atom or a lower alkyl radical, and also optical and geometrical isomers of said compounds of formula (I) and also their salts in the case where X—Y are each a bond of formula (a) and W is an —NH— radical optionally substituted by a lower alkyl radical.

The expression “lower alkyl radical” means a linear or branched alkyl radical having from 1 to 6 carbon atoms.

Among the compounds of formula (I) which may be included in the compositions of the present invention, especially representative are the following:
1. 6-[3-(3,4-bishydroxymethylbenzyloxy)phenyl]-2-methylhepta-3,5-dien-2-ol;
2. 7-[3-(3,4-bishydroxymethylphenoxymethyl)phenyl]-3-ethyloctan-3-ol;
3. 7-{3-[2-(3,4-bishydroxymethylphenyl)ethyl]phenyl}-3-ethylocta-4,6-dien-3-ol;
4. 6-{3-[2-(3,4-bishydroxymethylphenyl)ethyl]phenyl}-2-methyl hepta-3,5-dien-2-ol;
5. 7-{3-[2-(3,4-bishydroxymethylphenyl)vinyl]phenyl}-3-ethylocta-4,6-dien-3-ol;
6. 7-[3-(3,4-bishydroxymethylbenzyloxy)phenyl]-3-ethyl-3-octanol;
7. 4E,6E)-7-[3-(3,4-bishydroxymethylbenzyloxy)phenyl]-3-ethylocta-4,6-dien-3-ol;
8. (4E,6 E)-7-[3-(3,4-bishydroxymethyl benzyloxy)phenyl]-3-ethylnona-4,6-dien-3-ol;
9. (E)-7-[3-(3,4-bishydroxymethylbenzyloxy)phenyl]-3-ethyloct-4-en-3-ol;
10. (E)-7-[3-(3,4-bishydroxymethylbenzyloxy)phenyl]-3-ethyloct-6-en-3-ol;
11. (E)-7-[3-(3,4-bishydroxymethylbenzyloxy)phenyl]-3-ethyloct-6-en-4-yl-3-ol;
12. (4E,6E)-7-[3-(3,4-bishydroxymethylphenoxymethyl)phenyl]-3-ethylocta-4,6-dien-3-ol;
13. (E)-7-[3-(3,4-bishydroxymethylphenoxymethyl)phenyl]-3-ethylnon-6-en-3-ol;
14. (E)-7-{3-[(3,4-bishydroxymethylbenzyl)methylamino]phenyl}-3-ethyloct-6-en-3-ol; and
15. 7-[3-(3,4-bishydroxymethylbenzyloxy)phenyl]-3-ethyl-7-methyloctan-3-ol.

In particular, the pharmaceutical active principle incorporated into the compositions according to the invention is (4E,6E)-7-[3-(3,4-bishydroxymethylbenzyloxy)phenyl]-3-ethylnona-4,6-dien-3-ol.

Compositions of the Invention:

The compositions of the invention are preferably in the form of an emulsion.

The term “emulsion” is understood to be a composition comprising at least two phases having different polarities, in which at least one phase is dispersed in another phase.

The compositions of the present invention are “simple” emulsions such as water-in-oil (W/O), oil-in-water (O/W), water-in-silicone or glycol-in-silicone emulsions, or cream gels (oily phase dispersed in an aqueous phase by a non-surfactant polymeric emulsifier) or triple emulsions (for example W/O/W or O/W/O).

The compositions according to the invention are preferably emulsions that have at least one hydrophilic phase and at least one lipophilic phase, or else at least one glycolic or hydroglycolic phase and at least one lipophilic phase.

The proportions of each of the phases are selected as a function of the active principle and of its solubility in the hydrophilic phase and in the lipophilic phase, and this being in order to attain the active principle concentration that is therapeutically effective for the targeted pathology or pathologies.

According to the invention, the hydrophilic phase is 10% to 90% (expressed by weight relative to the total weight of the composition), preferably 10% to 45% of the composition.

According to the invention, the lipophilic phase is 10% to 90% (expressed by weight relative to the total weight of the composition), preferably 15% to 50% by weight of the composition.

Hydrophilic Phase of the Composition:

The compounds of the hydrophilic phase are selected as a function of the solubility profile of the active principle that it is desired to dissolve therein.

The hydrophilic phase comprises at least one hydrophilic solvent, which may be selected, in particular, from water or a mixture of water and C1 to C4 alcohol(s), such as ethanol, isopropanol or butanol (hydroalcoholic solution), and also glycols or else mixtures thereof, especially glycol/water mixtures (hydroglycolic phase). Furthermore, it should be noted that the hydrophilic solvent may be exclusively glycolic. An example of emulsions that include such a hydrophilic phase is a glycol/silicone type emulsion.

Preferably, at least one hydrophilic phase of the composition according to the invention is a glycolic or hydroglycolic phase.

Preferably, at least one hydrophilic phase of the composition according to the invention comprises the active principle dissolved in at least one hydrophilic solvent, and at least one other hydrophilic compound. This other compound may be another hydrophilic solvent, or any other compound introduced into the hydrophilic phase.

The glycols that can be employed in the present invention include alkylene or polyalkylene glycols. Examples thereof include (C1 to C6) alkylene and polyalkylene glycols such as ethylene glycol, polyethylene glycols (2 to 20 monomers), propylene glycol, dipropylene glycol, butylene glycol, pentylene glycol and hexylene glycol.

Preferably, the glycols employed according to the invention are selected from propylene glycol or PEG 400.

Lipophilic Phase of the Composition:

The compounds of the lipophilic phase are also selected as a function of the solubility profile of the active principle that it is desired to dissolve therein.

The expression “lipophilic phase” means a phase containing, on the one hand, the active principle and, on the other hand, compounds that are exclusively lipophilic. Thus, such a phase does not comprise any hydrophilic compound.

The lipophilic phase comprises at least one lipophilic solvent, which may be an oil, optionally a volatile oil. Preferably, it comprises at least one (optionally volatile) oil that is a solvent for the molecule and/or a wax.

Preferably, at least one lipophilic phase of the compositions according to the invention comprises the active principle dissolved in at least one lipophilic solvent, and at least one other lipophilic compound. This other compound may be another lipophilic solvent, or any other compound introduced into the lipophilic phase.

According to the invention, the wax may be an animal, plant, mineral or synthetic wax.

Among the animal waxes, exemplary are beeswax and whale blubber. Among the plant waxes, exemplary are carnauba wax, candelilla wax, ouricury wax, cork fiber waxes, sugarcane waxes and Japan waxes. Among the mineral waxes, exemplary are paraffin waxes, microcrystalline waxes, lignite waxes and ozokerites. Lastly, among the synthetic waxes, exemplary are polyethylene waxes and waxes obtained by Fischer-Tropsch synthesis.

Exemplary oils (lipophilic solvent) according to the present invention include mink oil, turtle oil, soybean oil, grapeseed oil, sesame oil, maize oil, rapeseed oil, sunflower oil, cottonseed oil, avocado oil, olive oil, castor oil, jojoba oil and peanut oil; hydrocarbon oils such as liquid paraffin, squalane and liquid petrolatum; esters such as isopropyl myristate, isopropyl palmitate, butyl stearate, hexyl laurate, isononyl isononanoate, cetearyl isononanoate, 2-ethylhexyl palmitate, 2-hexyldecyl laurate, 2-octyldecyl palmitate, 2-octyldodecyl myristate, 2-diethylhexyl succinate, diisostearyl malate, 2-octyldodecyl lactate, glyceryl triisostearate, caprylic/capric triglyceride, C₁₂₋₁₅ alkyl benzoate, perfluorinated and/or organofluorinated oils; higher fatty acids such as myristic acid, palmitic acid, stearic acid, behenic acid, oleic acid, linoleic acid, linolenic acid, isostearic acid, higher fatty alcohols such as cetyl alcohol, stearyl alcohol, oleyl alcohol, Guerbet alcohols such as hexyldecanol and octyldodecanol. The oils employed according to the invention may also be silicone-based oils of linear siloxane type and more preferably hexamethyldisiloxane. Exemplary is the product marketed by Dow Corning, DC Fluid® 0.65 cSt.

The components of the lipophilic phase according to the invention are preferably selected from among isopropyl palmitate, caprylic/capric triglyceride (MIGLYOL 812), C12-C15 alkyl benzoate (FINSOLV TN), cetearyl isononanoate (CETIOL SN) and octyldodecanol (EUTANOL G).

Surfactants are also advantageously used to obtain a stable emulsion. They are also preferably used to obtain very fine emulsions, especially in the case of multiple emulsions.

Surfactants (emulsifiers or surface-active agents) are natural or synthetic substances formed from a hydrophilic or polar part and from a lipophilic or apolar part. These are amphiphilic molecules since they have a double polarity. The surfactants are characterized by their HLB. The surfactants go to the interface of the hydrophilic and lipophilic phases. The HLB value of the surfactant orients the direction of the emulsion.

Preferably, the compositions in the form of emulsions according to the invention comprise at least one pharmacologically or cosmetologically acceptable surfactant that does not interact with the active principle, preferably present in the continuous phase of the emulsion.

Thus, one or more surfactant(s) with high HLB (for example, 15-16) are incorporated into the hydrophilic phase of the emulsion in order to prepare an O/W emulsion and may contribute to the solubilization of the active principle incorporated in the hydrophilic phase.

Similarly, one or more surfactants with low HLB (5-6) are incorporated into the lipophilic phase of the emulsion in order to prepare a W/O emulsion and may contribute to the solubilization of the active principle in the lipophilic phase.

The surfactants employed in the present invention must be pharmacologically or cosmetologically acceptable and must not interact with the active principle. Generally, the surfactants are incorporated, before emulsification, into the phase intended to form the continuous phase of the emulsion.

Exemplary non-ionic surfactants with high HLB include glycerol esters, polyoxyethylene glycol esters or polyoxypropylene glycol esters, esters of saccharose and fatty acids, polyoxyethylenated sorbitan esters and surfactants with an ether-oxide bond such as polyoxyethylenated or polyoxypropylenated alcohols.

Preferably, for producing oil-in-water type emulsions according to the invention, polyoxyethylene glycol esters are used such as PEG 100 stearate (ARLACEL® 165) or polyoxyethylenated alcohols such as ceteareth-20 (EUMULGIN® B2).

Exemplary non-ionic surfactants with low HLB include glycol esters, sorbitan esters and polyoxyethylenated alcohols.

Preferably, for producing water-in-oil type emulsions according to the invention, sorbitan esters such as sorbitan laurate, sorbitan oleate and sorbitan stearate are employed.

Exemplary silicone-based surfactants that make it possible to formulate silicone-based emulsions, especially water-silicone or glycol-silicone emulsions, include the surfactants:

of polyalkyl methicone copolyol type (optionally crosslinked oxyalkylenated polyalkylmethylsiloxane) containing linear or branched, saturated or unsaturated C₆ to C₂₀ alkyl chains, a polyoxyethylenated unit with 1 to 50 EO (ethylene oxide) and/or a polyoxypropylenated unit having 1 to 50 PO (propylene oxide); and

of oxyalkylenated polyalkyldimethylmethylsiloxane type containing: linear or branched, saturated or unsaturated C₆ to C₂₀ alkyl chains, a polyoxyethylenated unit having 1 to 50 EO and/or a polyoxypropylenated unit having 1 to 50 PO.

Advantageously, use will therefore be made, as silicone-based emulsifiers, of alkyl dimethicone copolyols such as ABIL EM-90, or the dimethicone copolyol/cyclomethicone mixture marketed by Dow Corning under the trademark 3235C FORMULATION AID, the lauryl methicone copolyol marketed under the trademark EMULSIFIER 10 by Dow Corning, or mixtures based on a silicone-based polymer such as cetyl dimethicone copolyol with polyglyceryl-4 isostearate and hexyl laurate marketed under the trademark ABIL WE09 by Goldschmidt, ABIL EM 97 by Goldschmidt (dimethicone copolyol & cyclomethicone), WACKER SPG 128 VP by Wacker (cyclomethicone and octyldimethicone methoxy glycosyl), SILWAX WD-IS (dimethicone copolyol isostearate). Non-polymeric silicone-based emulsifiers could also be used such as mono- or polyalkyl ester siloxanes, for example SILWAX S from Lambent (dimethiconol stearate) or alkoxylated carboxylic acid esters such as the polyhydroxylated alkyl esters of PEG, for example ARLACEL P 135 from Uniqema (PEG-30 dipolyhydroxystearate).

The formulation of the emulsions within a pharmaceutical or cosmetic composition employs the usual techniques of one skilled in the art, in the formulation and galenic field.

The compositions according to the invention are preferably suitable for topical application to the skin, integuments and/or mucous membranes, whether regime or regimen therefor. The compositions comprise a sufficient amount of active principle to obtain the desired effect.

Other compounds may be added to adjust the skin penetration, whether this is to improve it or to reduce it.

Among these additional compounds that adjust the skin penetration of the active principle, exemplary are hydrophilic absorption promoters, lipophilic absorption promoters and agents that limit the skin penetration by trapping the molecule.

Among the hydrophilic absorption promoters, exemplary are propylene glycol, transcutol, DMSO, ethanol, N-methylpyrrolidone and preferably propylene glycol.

Among the lipophilic absorption promoters, exemplary are oleic acid, oleyl alcohol, oleyl oleate, oleyl erucate, macadamia oil or alpha-bisabolol, terpenes, azone and preferably oleic acid.

Among the agents that limit skin penetration of the active principle, exemplary are polyethylene glycols, and preferably PEG 400. These compounds, through their network structure, are known to trap certain molecules in this network and slow down their release from the network to the target tissue.

The present invention may additionally comprise any additive normally used in the cosmetic or pharmaceutical field, such as sequestrants, wetting agents, adhesion promoters, spreading agents, antioxidants, sunscreens, preservatives, fillers, electrolytes, humectants, pigments, dyes, common mineral or organic bases or acids, essential oils, cosmetic active agents, moisturizers, vitamins, essential fatty acids and/or sphingolipids.

Of course, this or these optional additional compounds, and/or their amounts, are selected such that the advantageous properties of the emulsion are not, or are not substantially, impaired.

Manufacture of the Composition:

The present invention also features a method for formulating a cosmetic or pharmaceutical composition such as described above.

Two different methods are however distinguished as a function of the temperature sensitivity of the active principle. The expression “not temperature sensitive” means an active principle that withstands temperatures from 40° to 80° C. without undergoing physical and/or chemical degradation.

Method A:

In the case of an active principle that is not temperature sensitive, it may be heated with the other constituents of the various phases. The steps of the method are the following:

i) mixing of said active principle with at least one hydrophilic solvent at a temperature ranging from 40° C. to 80° C., in order to obtain a hydrophilic phase in which the active principle is present in dissolved form;

ii) mixing of said active principle with at least one lipophilic solvent at a temperature ranging from 40° C. to 80° C., in order to obtain a lipophilic phase in which the active principle is present in dissolved form; and

iii) mixing of the hydrophilic phase obtained in i) with the lipophilic phase obtained in ii).

Preferably, the method comprises, at the end of step ii), the addition of at least one other lipophilic compound to said lipophilic phase.

Said method thus comprises, in this case, the following steps:

i) mixing of said active principle with at least one hydrophilic solvent at a temperature ranging from 40° C. to 80° C., in order to obtain a hydrophilic phase in which the active principle is present in dissolved form;

ii) mixing of said active principle with at least one lipophilic solvent at a temperature ranging from 40° C. to 80° C., in order to obtain a lipophilic active phase in which the active principle is present in dissolved form, then mixing of this lipophilic active phase with at least one other lipophilic compound, in order to obtain a lipophilic phase; and

iii) mixing of at least one hydrophilic phase obtained in i) with at least one lipophilic phase obtained in ii).

Advantageously, the process according to the invention comprises the addition of at least one surfactant of suitable HLB during step i) or step ii). A surfactant with high HLB will be introduced into the hydrophilic phase, whereas a surfactant with low HLB will be introduced into the lipophilic phase.

Thus, preferably, the method for preparing the compositions according to the invention comprises the following steps:

1—Preparation of the Hydrophilic Phase:

a) Weighing the water and heating, preferably from 60° to 80° C., with gentle stirring.

b) Adding the active principle, the preservative, the chelating agent and the pH adjuster and where appropriate the surfactant with high HLB and stirring until these compounds have completely dissolved.

c) Incorporating the thickener and stirring, preferably for 20 to 40 minutes, at a temperature from 60° C. to 80° C., preferably at a temperature of 75° C.

d) Then adding the agent or agents for stabilizing the emulsion and leaving them stirring until completely dissolved.

2—Preparation of the Lipophilic Phase:

a) Weighing all the constituents of the lipophilic phase (except the active principle) and, in the case where the surfactant with high HLB is not incorporated into the hydrophilic phase, the surfactant with low HLB, and heating, preferably ranging from 60° C. to 80° C. while stirring, until a homogeneous phase is obtained.

b) The active principle, in its amount intended for this phase, is then added to the lipophilic phase and maintained under stirring until it has completely dissolved.

In one preferred embodiment of the method according to the invention, the active principle is dissolved prior to the production of phase a) above in a lipophilic solvent for the active principle, in order to obtain a mixture comprising the active principle. Step b) then entails adding this mixture comprising the active principle in its amount intended for this phase, to the lipophilic phase and maintained under stirring until it has completely dissolved.

3—Emulsification:

Pouring the hydrophilic phase into the lipophilic phase and emulsifying with vigorous stirring for a time from 5 and 20 minutes, preferably for 10 minutes.

Method B:

In the case of an active principle that is temperature sensitive, the method will have to be modified to avoid, as much as possible, heating the active principle to temperatures above 40° C. When the heating steps are necessary, for example in order to make constituents that are solid at ambient temperature liquid, then the contact time of the active principle with the phase at a temperature above 40° C. will be reduced to a minimum.

The steps of the method are the following:

i) mixing of said active principle with at least one hydrophilic solvent at a temperature below 40° C., in order to obtain a hydrophilic active phase in which the active principle is present in dissolved form;

ii) preparation of a hydrophilic non-active phase by heating at least one other hydrophilic compound at a temperature ranging from 40° C. to 80° C.;

iii) mixing of said active principle with at least one lipophilic solvent at a temperature below 40° C., in order to obtain a lipophilic phase in which the active principle is present in dissolved form;

iv) mixing of the hydrophilic non-active phase obtained in ii) with the lipophilic phase obtained in iii) at a temperature of around 50° C. to 60° C., in order to obtain an emulsion;

v) cooling the emulsion obtained in iv) down to a temperature of 30° C. to 45° C.; and

vi) mixing of said emulsion obtained in v) with said hydrophilic active phase obtained in i) at a temperature ranging from 30° C. to 45° C.

Advantageously, the method according to the invention comprises the addition of at least one surfactant of suitable HLB during step ii) (preparation of the hydrophilic non-active phase) or step iii) (preparation of the lipophilic phase).

Preferably, the method comprises the following steps:

i) mixing of said active principle with at least one hydrophilic solvent at a temperature below 40° C., in order to obtain a hydrophilic active phase in which the active principle is present in dissolved form;

ii) preparation of a hydrophilic non-active phase by heating at least one other hydrophilic compound at a temperature ranging from 40° C. to 80° C.;

iii) mixing of said active principle with at least one lipophilic solvent at a temperature below 40° C., in order to obtain a lipophilic active phase in which the active principle is present in dissolved form;

iv) preparation of a lipophilic non-active phase by heating at least one other lipophilic compound at a temperature ranging from 40° C. to 80° C.;

v) cooling the lipophilic non-active phase obtained in iv) down to a temperature of at most 40° C.;

vi) mixing of the cooled lipophilic non-active phase obtained in v) with the lipophilic active phase obtained in iii);

vii) mixing of the hydrophilic non-active phase obtained in ii) with the lipophilic phase obtained in vi) at a temperature of around 50° C. to 60° C., in order to obtain an emulsion;

viii) cooling the emulsion obtained in vii) down to a temperature of 30° C. to 45° C.; and

ix) mixing of said emulsion obtained in viii) with said hydrophilic active phase obtained in i) at a temperature ranging from 30° C. to 45° C.

Advantageously, the method according to the invention comprises the addition of at least one surfactant of suitable HLB during step ii) (preparation of the hydrophilic non-active phase) or step iv) (preparation of the lipophilic non-active phase). A surfactant with high HLB will be introduced into the hydrophilic phase, whereas a surfactant of low HLB will be introduced into the lipophilic phase.

Thus, preferably, the method for preparing the composition according to the invention comprises the following steps:

1—Preparation of the Hydrophilic Phase:

a) Weighing the water and heating, preferably ranging from 60° C. to 80° C., with gentle stirring.

b) Adding the preservative, the chelating agent and the pH adjuster and where appropriate the surfactant with high HLB and stirring until these compounds have completely dissolved.

c) Incorporating the thickener and stirring, preferably for 20 to 40 minutes, at a temperature ranging from 60° C. to 80° C., preferably at a temperature of 75° C.

d) Then adding the agent or agents for stabilizing the emulsion and leaving them stirring until completely dissolved.

e) Furthermore, dissolving the amount of active principle intended for the hydrophilic phase in a glycol/antioxidant mixture at a temperature below 40° C., preferably at ambient temperature.

2—Preparation of the Lipophilic Phase:

a) Identifying the constituents of the lipophilic phase which are liquid at ambient temperature and those that are solid which need to be melted.

b) Identifying at least one of the constituents of the lipophilic phase that is liquid at ambient temperature and is a solvent for the active principle, and dissolving the active principle therein.

c) Weighing all the other constituents of the lipophilic phase and, in the case where the surfactant with high HLB is not incorporated into the hydrophilic phase, the surfactant with the low HLB, and heating, preferably at a temperature necessary for melting the solid constituents, namely at a temperature ranging from 60° C. to 80° C., with stirring, until a homogeneous phase is obtained. Preferably, letting the temperature of the phase cool as much as possible before the next step.

d) Adding the active principle/lipophilic ingredient mixture to this lipophilic phase and leaving stirring until a new homogeneous phase has been obtained while minimizing the contact time of the active principle with the potentially high temperature of the lipophilic phase. Proceeding rapidly to the following emulsification and cooling.

3—Emulsification

a) Pouring the hydrophilic phase obtained at the end of step d) from the preparation of the hydrophilic phase into the lipophilic phase and emulsifying with vigorous stirring for a time from 5 to 20 minutes, preferably for 10 minutes, at a temperature of around 50° C. to 60° C.

b) Slowly cooling, while continuing to stir the emulsion at a moderate rate.

c) At a temperature ranging from 30° C. to 45° C., and preferably around 35° C.-40° C., incorporating the active principle intended for the hydrophilic phase that has previously been dissolved in the glycol/antioxidant mixture (step e) of the preparation of the hydrophilic phase) into the emulsion and continuing the stirring until the emulsion returns to ambient temperature.

In the preceding, stirring of the solutions or of the emulsions according to the present description may be carried out by means of conventional devices, such as, for example, a Rayneri device.

The expression “gentle stirring” means, according to the invention, stirring obtained from a Rayneri device having a value below 300 rpm.

The expression “moderate stirring” means, according to the invention, stirring obtained from a Rayneri device from 301 to 700 rpm.

The expression “vigorous stirring” means, according to the invention, stirring obtained from a Rayneri device from 701 to 1500 rpm.

The expression “ambient temperature” means a temperature ranging from 20° C. to 30° C., preferably ranging from 23° C. to 27° C., preferably a temperature equal to 25° C.

According to one particularly preferred embodiment, the composition prepared according to the method of the invention is an oil-in-water (O/W) type emulsion, in other words an emulsion in which said first phase according to the description is a lipophilic phase dispersed in a second continuous, hydrophilic phase.

Cosmetic or Pharmaceutical Applications:

The compositions according to the invention may be used for a pharmaceutical or cosmetic application. They are preferably in a form suitable for topical applications, for example in the form of a cream, mousse, spray or aerosol or in any administration form that is compatible with the claimed formulation.

Preferably, the compositions according to the invention contain vitamin D or a vitamin D derivative and are applicable to the field of dermatology, for example in the applications indicated below, preferably in the treatment of psoriasis.

This composition is particularly suitable for, whether regime or regimen:

1) treating dermatological conditions or afflictions linked to a differentiation or proliferation disorder of keratinocytes or sebocytes, especially for treating common acne, comedone-type acne, polymorphic acne, acne rosacea, nodulocystic acne, acne conglobata, senile acne, secondary acne such as solar acne, acne medicamentosa or occupational acne;

2) treating keratinization disorders, in particular ichthyosis, ichthyosiform states, Darier's disease, palmoplantar keratoderma, leukoplasia and leukoplasiform states, cutaneous or mucosal (buccal) lichen;

3) treating other dermatological conditions linked to a keratinization disorder with an inflammatory and/or immunoallergic component and, especially, all forms of psoriasis, whether cutaneous, mucous or ungual, and even psoriatic rheumatism, or else skin atopy such as eczema or a respiratory atopy or else gingival hypertrophy;

4) treating certain inflammatory skin conditions that do not exhibit a keratinization disorder, such as atopic eczema and contact allergy;

5) treating any dermal or epidermal proliferations whether benign or malignant, whether of viral or non-viral origin such as verrucae vulgaris, verrucae plana and epidermodysplasia verruciformis, oral or florid papillomatoses, and proliferations which may be induced by ultraviolet radiation, especially in the case of baso- and spinocellular epitheliomas;

6) treating other dermatological disorders such as bullous dermatoses and collagen diseases;

7) preventing or treating the signs of skin aging, whether photoinduced or chronological or for reducing actinic keratoses and pigmentations, or any skin pathologies associated with chronological or actinic aging;

8) preventing or treating cicatrization disorders or for preventing or repairing stretch marks;

9) combating sebaceous function disorders such as acne hyperseborrhea or simple seborrhea or even seborrheic eczema;

10) treating certain opthalmalogical disorders, especially corneopathies;

11) the treatment or prevention of cancerous or precancerous states of skin cancers or other cancers presenting, or which may be induced to present, vitamin D receptors, such as, but non-limitingly, breast cancer, leukemia, myelodysplastic syndromes and lymphomas, carcinomas of malpighian epithelium cells and gastrointestinal cancers, melanomas, and osteosarcoma;

12) the treatment of inflammatory conditions such as arthritis or rheumatoid arthritis;

13) the treatment of any cutaneous or general condition of viral origin;

14) the prevention or treatment of alopecia of various origins, especially alopecia due to chemotherapy or radiation;

15) the treatment of dermatological or general conditions having an immunological component;

16) the treatment of immune conditions, such as autoimmune diseases (such as, but non-limitingly, type 1 diabetes, multiple sclerosis, lupus and lupus-type conditions, asthma, glomerulonephritis, etc.), selective dysfunctions of the immune system (for example, AIDS) and the prevention of immune rejection, such as the rejection of transplants (for example, kidney, heart, bone marrow, liver, Langerhans' islets or the whole of the pancreas, skin, etc.) or the prevention of graft-versus-host disease;

17) the treatment of endocrine conditions which may be treated by vitamin D analogues such as those that advantageously modulate hormonal secretion, for example by increasing the secretion of insulin or selectively suppressing the secretion of the parathyroid hormone (for example, in chronic renal failure and secondary hyperparathyroidism);

18) the treatment of conditions characterized by abnormal intracellular calcium handling; and

19) the treatment and/or the prevention of vitamin D deficiencies and other conditions of mineral homeostasis in plasma and bone, such as rickets, osteomalacia, osteoporosis, especially in the case of menopausal women, renal osteodystrophy and parathyroid function disorders.

One particular embodiment of the invention is therefore an emulsion comprising a hydrophilic phase and a lipophilic phase, vitamin D or a vitamin D analogue or derivative, i.e., preferably (4E,6E)-7-[3-(3,4-bishydroxymethylbenzyloxy)phenyl]-3-ethylnona-4,6-dien-3-ol being present and in dissolved form in each of these phases.

One embodiment of the invention more particularly relates to an emulsion comprising a glycolic or hydroglycolic phase and a lipophilic phase, (4E,6E)-7-[3-(3,4-bishydroxymethylbenzyloxy)phenyl]-3-ethylnona-4,6-dien-3-ol being present in dissolved form in each of these phases.

Another embodiment of the invention features pharmaceutical or cosmetic compositions for topical application comprising such an emulsion.

Another embodiment of the invention features the use of a composition such as defined above, especially an emulsion, for preparing a medication useful for the treatment of skin disorders, especially psoriasis.

This is because vitamin D and its analogues or derivatives limit the excessive production of skin cells on the affected surfaces and have proven advantages for the treatment of this condition which is characterized in particular by the presence of thick, scaly and dry lesions.

In order to further illustrate the present invention and the advantages thereof, the following specific examples are given, it being understood that same are intended only as illustrative and in nowise limitative.

In Examples 1 and 2, the amounts of the constituents incorporated into the composition of the formulations are expressed in weight percentages relative to the total weight of the composition.

### EXAMPLES

### Example 1

### Manufacturing Method that can be Used for Examples 2 or 3

1—Preparation of the Hydrophilic Phase:

Weigh the water and heat to 75° C., with gentle stirring.

Add EDTA, sodium hydrogen phosphate and methylparaben and stir at a moderate rate until completely dissolved.

Incorporate VEEGUM K and stir at a moderate rate for 30 minutes, at 75° C.

Next, add KELTROL T and leave stirring until completely dissolved.

2—Preparation of the Lipophilic Phase:

Weigh all the constituents of the lipophilic phase (except for (4E,6E)-7-[3-(3,4-bishydroxymethylbenzyloxy)phenyl]-3-ethylnona-4,6-dien-3-ol) and heat to 75° C. with moderate stirring, until a homogeneous phase is obtained.

Add the (4E,6E)-7-[3-(3,4-bishydroxymethylbenzyloxy)phenyl]-3-ethylnona-4,6-dien-3-ol intended for this phase and leave stirring until completely dissolved.

3—Emulsification:

Pour the hydrophilic phase into the lipophilic phase and emulsify with vigorous stirring (1200 rpm) for 10 minutes at a temperature of around 60° C.

Slowly cool while maintaining moderate stirring at 700 rpm.

At around 35-40° C., incorporate the (4E,6E)-7-[3-(3,4-bishydroxymethylbenzyloxy)phenyl]-3-ethylnona-4,6-dien-3-ol previously dissolved in the glycol/BHA mixture into the emulsion and continue stirring until the emulsion returns to ambient temperature.

### Example 2

### O/W Emulsion

| Ingredients | | Role in the | |
|---|---|---|---|
| INCI name | Trademark | emulsion | % |
| LIPOPHILIC | | | |
| Phase: | | | |
| Ceteareth-20 | EUMULGIN B2 | Surfactant | 3.75 |
| Glyceryl | CUTINA GMS-V | Consistency factor | 6.25 |
| stearate | | | |
| Caprylic/capric | MIGLYOL 812 | Emollient | 15 |
| triglyceride | | Solvent for the active | |
| | | principle | |
| Isopropyl | CRODAMOL IPP | Emollient | 10 |
| palmitate | | Solvent for the active | |
| | | principle | |
| Vaseline | VASELINE VARA | Occlusive agent | 3 |
| | 5718 | | |
| Dimethicone | Silicone DC 200, | Spreading agent | 1 |
| | 350 cSt | | |
| Propylparaben: | Propyl paraben | Preservative | 0.05 |
| Propyl | | | |
| parahydroxybenzoate | | | |
| C₁₂₋₁₅ alkyl | TEGOSOFT TN | Emollient | 12 |
| benzoate | | Solvent for the active | |
| | | principle | |
| Tocopherol | DL-alpha | Antioxidant | 0.04 |
| | tocopherol | | |
| (4E,6E)-7-[3- | (4E,6E)-7-[3-(3,4- | Active principle | 0.1 |
| (3,4-Bishydroxymethylbenzyloxy)phenyl]- | bishydroxymethylbenzyloxy)phenyl]- | | |
| 3-ethylnona-4,6- | 3-ethylnona-4,6- | | |
| dien-3-ol | dien-3-ol | | |
| HYDROPHILIC | | | |
| Phase: | | | |
| Xanthan gum | KELTROL T | Thickener/stabilizer | 0.22 |
| Methylparaben | Methyl paraben | Preservative | 0.2 |
| Magnesium | VEEGUM K | Thickener/stabilizer | 0.8 |
| aluminum | | | |
| silicate | | | |
| Polyethylene | PEG 400 | Solvent for the active | 20 |
| glycol 400 | | principle | |
| BHA | BHA | Antioxidant | 0.01 |
| (butylhydroxy | | | |
| anisole) | | | |
| Disodium EDTA | EDTA 2Na | Chelating agent | 0.1 |
| Disodium | Na₂HPO₄, 2H₂O | pH adjuster | 0.4 |
| hydrogen | | | |
| phosphate | | | |
| dihydrate | | | |
| Purified water | Purified water | Solvent | qs for |
| | | | 100 |
| (4E,6E)-7-[3- | (4E,6E)-7-[3-(3,4- | Active principle | 0.2 |
| (3,4-Bishydroxymethylbenzyloxy)phenyl]- | bishydroxymethylbenzyloxy)phenyl]- | | |
| 3-ethylnona-4,6- | 3-ethylnona-4,6- | | |
| dien-3-ol | dien-3-ol | | |

### Example 3

### O/W Emulsion

| Ingredients | | Role in the | % |
|---|---|---|---|
| INCI name | Trademark | emulsion | % |
| LIPOPHILIC | | | |
| Phase: | | | |
| Ceteareth-20 | EUMULGIN B2 | Surfactant | 3.75 |
| Glyceryl | CUTINA GMS-V | Consistency factor | 6.25 |
| stearate | | | |
| Caprylic/capric | MIGLYOL 812 | Emollient | 15 |
| triglyceride | | Solvent for the active | |
| | | principle | |
| Isopropyl | CRODAMOL IPP | Emollient | 10 |
| palmitate | | Solvent for the active | |
| | | principle | |
| Vaseline | VASELINE VARA | Occlusive agent | 3 |
| | 5718 | | |
| Dimethicone | Silicone DC 200, | Spreading agent | 1 |
| | 350 cSt | | |
| Propylparaben: | Propyl paraben | Preservative | 0.05 |
| Propyl | | | |
| parahydroxybenzoate | | | |
| C₁₂₋₁₅ alkyl | TEGOSOFT TN | Emollient | 12 |
| benzoate | | Solvent for the active | |
| | | principle | |
| BHT: | BHT | Antioxidant | 0.04 |
| Butylhydroxy | | | |
| toluene | | | |
| (4E,6E)-7-[3- | (4E,6E)-7-[3-(3,4- | Active principle | 0.2 |
| (3,4-Bishydroxymethylbenzyloxy)phenyl]- | bishydroxymethylbenzyloxy)phenyl]- | | |
| 3-ethylnona-4,6- | 3-ethylnona-4,6- | | |
| dien-3-ol | dien-3-ol | | |
| HYDROPHILIC | | | |
| Phase: | | | |
| Xanthan gum | KELTROL T | Thickener/stabilizer | 0.22 |
| Methylparaben: | Methyl paraben | Preservative | 0.2 |
| Methyl | | | |
| parahydroxybenzoate | | | |
| Magnesium | VEEGUM K | Thickener/stabilizer | 0.8 |
| aluminum | | | |
| silicate | | | |
| Propylene glycol | Propylene glycol | Solvent for the active | 20 |
| | | principle | |
| BHA | BHA | Antioxidant | 0.01 |
| (butylhydroxyanisole) | | | |
| Hydroxypropyl | METHOCEL | Thickener/stabilizer | 0.2 |
| methyl cellulose | | | |
| Purified water | Purified water | Solvent | qs for |
| | | | 100 |
| (4E,6E)-7-[3- | (4E,6E)-7-[3-(3,4- | Active principle | 0.1 |
| (3,4-Bishydroxymethylbenzyloxy)phenyl]- | bishydroxymethylbenzyloxy)phenyl]- | | |
| 3-ethylnona-4,6- | 3-ethylnona-4,6- | | |
| dien-3-ol | dien-3-ol | | |

The above examples illustrate the advantage of the mixed solubilization.

This is because the preparation of a 0.3% emulsion of (4E,6E)-7-[3-(3,4-bishydroxymethylbenzyloxy)phenyl]-3-ethylnona-4,6-dien-3-ol has been made possible solely due to a mixed solubilization of the molecule: one fraction in the hydrophilic phase and one fraction in the lipophilic phase.

Solubilization tests on the single phase (4E,6E)-7-[3-(3,4-bishydroxymethylbenzyloxy)phenyl]-3-ethylnona-4,6-dien-3-ol (in the hydrophilic phase or in the lipophilic phase) have not made it possible to obtain an emulsion that was physically stable over time; crystal growth phenomena of the (4E,6E)-7-[3-(3,4-bishydroxymethylbenzyloxy)phenyl]-3-ethylnona-4,6-dien-3-ol appeared from the first month of storage at 4° C.

On the other hand, the mixed solubilization enabled a good physical stability of the emulsion and the absence of crystal growth after 3 months of storage at 4° C.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A physically stable pharmaceutical/cosmetic composition which comprises:
at least one hydrophilic phase;
at least one lipophilic phase, comprising at least one lipophilic solvent; and
at least one active principle,
wherein the logarithm of the partition coefficient of the active principle ranges from 2 to 6, and in that the active principle is present in dissolved state in at least one hydrophilic phase and also in at least the lipophilic solvent of at least one lipophilic phase.

2. The pharmaceutical/cosmetic composition as defined by claim 1, wherein said at least one hydrophilic phase comprises the active principle dissolved in at least one hydrophilic solvent, and at least one other hydrophilic compound.

3. The pharmaceutical/cosmetic composition as defined by claim 1, formulated as an emulsion.

4. The pharmaceutical/cosmetic composition as defined by claim 1, comprising a single hydrophilic phase and a single lipophilic phase.

5. The pharmaceutical/cosmetic composition as defined by claim 1, wherein said at least one hydrophilic phase comprises a glycolic or hydroglycolic phase.

6. The pharmaceutical/cosmetic composition as defined by claim 1, wherein said at least one active principle is selected from the group consisting of steroidal anti-inflammatory agents, anti-fungal agents, anti-parasitic agents, nuclear receptor modulators and sex steroids.

7. The pharmaceutical/cosmetic composition as defined by claim 6, in which the at least one active principle comprises a corticosteroid.

8. The pharmaceutical/cosmetic composition as defined by claim 6, in which the at least one active principle comprises a retinoid.

9. The pharmaceutical/cosmetic composition as defined by claim 6, in which the at least one active principle comprises a vitamin D derivative.

10. The pharmaceutical/cosmetic composition as defined by claim 6, in which the at least one active principle comprises (4E,6E)-7-[3-(3,4-bishydroxymethylbenzyloxy)phenyl]-3-ethylnona-4,6-dien-3-ol.

11. The pharmaceutical/cosmetic composition as defined by claim 1, comprising at least one pharmacologically or cosmetologically acceptable surfactant.

12. The pharmaceutical/cosmetic composition as defined by claim 1, formulated for topical application.

13. The pharmaceutical/cosmetic composition as defined by claim 1, in which the at least one active principle is selected from the group consisting of:
6-[3-(3,4-bishydroxymethylbenzyloxy)phenyl]-2-methylhepta-3,5-dien-2-ol;
7-[3-(3,4-bishydroxymethylphenoxymethyl)phenyl]-3-ethyloctan-3-ol;
7-{3-[2-(3,4-bishydroxymethylphenyl)ethyl]phenyl}-3-ethylocta-4,6-dien-3-ol;
6-{3-[2-(3,4-bishydroxymethylphenyl)ethyl]phenyl}-2-methylhepta-3,5-dien-2-ol;
7-{3-[2-(3,4-bishydroxymethylphenyl)vinyl]phenyl}-3-ethylocta-4,6-dien-3-ol;
7-[3-(3,4-bishydroxymethylbenzyloxy)phenyl]-3-ethyl-3-octanol;
4E,6E)-7-[3-(3,4-bishydroxymethylbenzyloxy)phenyl]-3-ethylocta-4,6-dien-3-ol;
(E)-7-[3-(3,4-bishydroxymethylbenzyloxy)phenyl]-3-ethyloct-4-en-3-ol;
(E)-7-[3-(3,4-bishydroxymethylbenzyloxy)phenyl]-3-ethyloct-6-en-3-ol;
(E)-7-[3-(3,4-bishydroxymethylbenzyloxy)phenyl]-3-ethyloct-6-en-4-yl-3-ol;
(4E,6E)-7-[3-(3,4-bishydroxymethylphenoxymethyl)phenyl]-3-ethylocta-4,6-dien-3-ol;
(E)-7-[3-(3,4-bishydroxymethylphenoxymethyl)phenyl]-3-ethylnon-6-en-3-ol;
(E)-7-{3-[(3,4-bishydroxymethylbenzyl)methylamino]phenyl}-3-ethyloct-6-en-3-ol; and
7-[3-(3,4-bishydroxymethylbenzyloxy)phenyl]-3-ethyl-7-methyloctan-3-ol.

14. The pharmaceutical/cosmetic composition as defined by claim 1, formulated as a cream, mousse, spray or aerosol.

15. The pharmaceutical/cosmetic composition as defined by claim 1, essentially devoid of recrystallized active principle.

16. The pharmaceutical/cosmetic composition as defined by claim 11, said at least one surfactant having an HLB ranging from 15 to 16.

17. The pharmaceutical/cosmetic composition as defined by claim 11, said at least one surfactant having an HLB ranging from 5 to 6.

18. A regime or regimen for the treatment of psoriasis, comprising administering to an individual in need of such treatment, a thus effective amount of the pharmaceutical/cosmetic composition as defined by claim 1.

19. A method for the formulation of a pharmaceutical/cosmetic composition as defined in claim 1, which comprises the following steps:
i) mixing said at least one active principle with at least one hydrophilic solvent at a temperature ranging from 40° C. to 80° C., to obtain a hydrophilic phase in which the at least one active principle is present in dissolved state;
ii) mixing said at least one active principle with at least one lipophilic solvent at a temperature from 40° C. to 80° C., to obtain a lipophilic phase in which the active principle is present in dissolved state; and
iii) mixing the hydrophilic phase obtained in i) with the lipophilic phase obtained in ii).

20. The method as defined by claim 19, which comprises, at the end of step ii), the addition of at least one other lipophilic compound to said lipophilic phase.

21. The method as defined by claim 19, comprising adding at least one surfactant of predetermined HLB during step i) or step ii).

22. A method for the formulation of a pharmaceutical/cosmetic composition as defined by claim 2, which comprises the following steps:
i) mixing said at least one active principle with at least one hydrophilic solvent at a temperature below 40° C., to obtain a hydrophilic active phase in which the at least one active principle is present in dissolved state;
ii) preparing a hydrophilic non-active phase by heating at least one other hydrophilic compound at a temperature ranging from 40° C. to 80° C.;
iii) mixing said at least one active principle with at least one lipophilic solvent at a temperature below 40° C., to obtain a lipophilic phase in which the at least one active principle is present in dissolved state;
iv) mixing the hydrophilic non-active phase obtained in ii) with the lipophilic phase obtained in iii) at a temperature of about 50° C. to 60° C., to obtain an emulsion;
v) cooling the emulsion obtained in iv) to a temperature of 30° C. to 45° C.; and
vi) mixing said emulsion obtained in v) with said hydrophilic active phase obtained in i) at a temperature ranging from 30° C. to 45° C.

23. A method for the formulation of a pharmaceutical/cosmetic composition as defined by claim 1, which comprises the following steps:
i) mixing said at least one active principle with at least one hydrophilic solvent at a temperature below 40° C., to obtain a hydrophilic active phase in which the at least one active principle is present in dissolved state;
ii) preparing a hydrophilic non-active phase by heating at least one other hydrophilic compound at a temperature ranging from 40° C. to 80° C.;
iii) mixing said at least one active principle with at least one lipophilic solvent at a temperature below 40° C., to obtain a lipophilic active phase in which the at least one active principle is present in dissolved state;
iv) preparing a lipophilic non-active phase by heating at least one other lipophilic compound at a temperature ranging from 40° C. to 80° C.;
v) cooling the lipophilic non-active phase obtained in iv) to a temperature of at most 40° C.;
vi) mixing at least one cooled lipophilic non-active phase obtained in v) with the lipophilic active phase obtained in iii);
vii) mixing the hydrophilic non-active phase obtained in ii) with the lipophilic phase obtained in vi) at a temperature of about 50° C. to 60° C., to obtain an emulsion;
viii) cooling the emulsion obtained in vii) to a temperature of 30° C. to 45° C.; and
ix) mixing said emulsion obtained in viii) with said hydrophilic active phase obtained in i) at a temperature ranging from 30° C. to 45° C.

24. The method as defined by claim 23, comprising adding at least one surfactant of predetermined HLB during step ii) or step iv).
